(19) United States
(12) Patent Application Publication
(10) Pub. No.: US 2012/0165315 A1
(43) Pub. Date: Jun. 28, 2012

(54) MEPTAZINOL CARBAMATE PRODRUG SALTS

(76) Inventors: Paul McGee, Baskinstoke (GB); R. Melling, Hampshire (GB); Jean-Luc Coudret, Hampshire (GB); A. Manage, Hampshire (GB); Jean-Francois Camiaux, Hampshire (GB); Colin Lindley, Hampshire (GB)

(21) Appl. No.: 13/335,927

(22) Filed: Dec. 22, 2011

Related U.S. Application Data
(60) Provisional application No. 61/427,087, filed on Dec. 23, 2010.

(30) Foreign Application Priority Data
Jul. 4, 2011 (GB) 1111383.4

(51) Int. Cl. A61K 31/55; A61P 25/00; C07D 223/04

(52) U.S. Cl. 514/212.01; 540/611

(57) ABSTRACT

The present invention relates to salts of meptazinol carbamate prodrugs, and to their synthesis and use.

### CROSS REFERENCE TO RELATED APPLICATIONS

This application claims priority under 35 U.S.C. 119(e) to U.S. Provisional Application No. 61/427,087 filed Dec. 23, 2010 and also claims the benefit of GB Provisional Application No. 1111383.4 filed Jul. 4, 2011.

### TECHNICAL FIELD

The present invention relates to salts of meptazinol carbamate prodrugs, and to their synthesis and use. The invention provides amongst other things salts of meptazinol carbamate prodrugs which aim to improve the meptazinol's systemic availability and/or minimize the adverse gastrointestinal (GI) side-effects associated with the administration of meptazinol.

### BACKGROUND OF THE INVENTION

Meptazinol para-amino benzoic acid carbamate and various analogues thereof are described in provisional patent application U.S. 61/292,362 filed Jan. 5, 2010, the contents of which are expressly incorporated herein by reference. Processes for manufacturing meptazinol para-amino benzoic acid carbamate and various analogues thereof are included in a provisional application No. 61/427,106 entitled “Process for the Production of Meptazinol p-amino-benzoyl carbamate” filed on Dec. 23, 2010, the contents of which are expressly incorporated herein by reference. The contents of U.S. Provisional Application No. 61/427,087 filed Dec. 23, 2010, GB Provisional Application No. 1111383.4 filed Jul. 4, 2011, and PCT/GB2011/052567 filed Dec. 22, 2011 are also incorporated herein by reference in their entirety.

Appropriate treatment of pain continues to represent a major challenge for both patients and healthcare professionals. Optimal pharmacological management of pain requires selection of the appropriate analgesic drug that achieves rapid efficacy with minimal side effects. Opioid analgesics offer perhaps the most important option in the treatment of nociceptive pain and remain the gold standard of treatment. However, misuse and abuse of opioids is a widespread problem and may deter physicians from prescribing these drugs.

While affording good pain relief, opioids are blighted by unwanted GI side-effects, for example, constipation, nausea and vomiting. It has been found that a significant number of patients would rather endure their pain than suffer the incapacitating effects of chronic constipation, an enlightening measure of the severity and distress that this problem causes (Vanegas (1998). Cancer Nursing 21, 289-297).

A further shortcoming of many opioids is that they suffer from poor oral bioavailability. This has been shown, for example, with oxymorphone (Sloan et al. (2005). Supp Care Cancer 13, 57-65), meptazinol (Norbury et al. (1983). Eur J Clin Pharmacol 25, 77-80) and buprenorphine (Kintz and Marquet (2002). pp 1-11 in Buprenorphine Therapy in Opiate Addiction, Humana press). The poor oral bioavailability results in variable blood levels of the respective opioid and, therefore, variable patient response—a highly undesirable feature in the treatment of pain where rapid and reliable relief is demanded.

Various types of prodrugs have historically been proposed to improve the oral bioavailability of opioids. These have included simple ester conjugates which are frequently hydrolyzed by plasma esterases extremely quickly. Such rapid hydrolysis by plasma esterases may limit the utility of ester linked prodrugs because it would not allow for transient protection of the opioid against first pass metabolism.

The rapidity of hydrolysis of ester conjugates is illustrated by work on the morphine ester prodrug morphine-3-propionate. Morphine has a poor oral bioavailability due to extensive first pass glucuronidation at the 3- and the 6-positions, resulting in much inter and intra subject variability in analgesic response after an oral dose of the drug (Hoskin (1989). Br. J. Clin Pharmacol 27, 499-505). The plasma and tissue stability of the 3-propionate prodrug was investigated, and it was found to be hydrolyzed in human plasma with a half-life of less than 5 minutes (Goth et al. (1997). International Journal of Pharmaceutics 154, 149-155).

Meptazinol is another opioid with poor oral bioavailability (<10%). The low oral bioavailability has been attributed to high first pass glucuronidation (Norbury et al. (1983) Eur. J. Clin. Pharmacol. 25, 77-80). Attempts have been made to solve this problem by using ester linked meptazinol prodrugs (Lu et al. (2005). Biorg. and Med. Chem. Letters 15, 2607-2609 and Xie et al., (2005). Biorg. and Med. Chem. Letters 15, 493-4956). However, only one of these prodrugs—((Z)-3-[2-(propionyloxy)phenyl]-2-propenoic ester) showed a significant increase in bioavailability over meptazinol itself, when tested in a rat model. However, to the Applicants knowledge, no further data has been published on this prodrug.

An alternative strategy for creating a prodrug from the hydroxylic/oxo functions present in the opioids is the formation of O-alkyl (alkyl ether) or aryl ether conjugates. However, such derivatives appear to be very resistant to hydrolysis and metabolic activation. This is best illustrated by the 3-methyl ether prodrug of morphine—codeine. While codeine was not originally developed as a prodrug of morphine, it was subsequently found to give rise to small quantities of morphine. It has been estimated that less than 5% of an oral dose of codeine is converted to morphine—reflecting the slowness with which O-dealkylation takes place (Vree at al. (1992). Biopharma Drug Dispos. 13, 445-460 and Quiding et al. (1993). Eur. J. Clin. Pharmacol. 44, 319-323). The same phenomenon was observed for the corresponding dihydromorphine prodrug—dihydrocodeine, with less than 2% of an oral dose of dihydrocodeine being converted to dihydromorphine (Balikova at al. (2001). J. Chromatog. Biomed. Sci. Appl. 752, 179-186).

A further disadvantage of the O-alkyl ether prodrugging strategy is that the dealkylation of these opioids is effected by cytochrome P450 2D6 (Cyp2D6), a polymorphically expressed enzyme (Schmidt at al. (2003). Int. J. Clin. Pharmacol. Ther. 41, 95-106). This inevitably results in substantial variation in patient exposure to the respective active metabolite (e.g., morphine and dihydromorphine). For example, low/negligible exposure to morphine derived from codeine has been reported amongst a large group of patients deficient in Cyp2D6 activity, potentially impacting the analgesic efficacy of codeine (Poulsen et al. (1998). Eur. Clin. Pharmacol. 54, 451-454).

An ideal prodrug moiety and linkage for a particular opioid would achieve the appropriate rate and site of cleavage, to form the active opioid compound. There remains a real need in the treatment of severe pain with opioids, for products which retain all the inherent pharmacological advantages of the opioids, but which avoid their principal limitations of (1) induction of adverse GI side effects, including chronic constipation; and (2) low and erratic systemic availability after oral dosing.

While the prodrug approach using meptazinol p-amino-benzoic acid carbamate and analogues thereof has been shown to work with the prodrug as a free base, the free base is very hygroscopic. Absorption of moisture leads to stability problems in pharmaceuticals. Therefore there is a need in the art for a more stable form of meptazinol p-amino-benzoic acid carbamate and analogues thereof.

One aim of the invention is to provide salts which are stable crystals. In particular, the invention seeks to provide salt which are stable to moisture and/or oxidation. Thus one aim is to provide salts which are not hygroscopic. Another aim is to provide salts which are not discoloured. It is also an aim to provide salts that can be obtained in as high or higher purity than the parent active starting material. It is a particular aim to provide salt forms having improved purity relative to the free base starting material. It is a further aim that the salts can be obtained in good yield. The present invention thus satisfies some or all of the above aims.

International Application No. PCT/GB2011/052567 in incorporated herein by reference in its entirety.

### SUMMARY OF THE INVENTION

In a first aspect the present invention provides salts of meptazinol prodrugs of formula I:

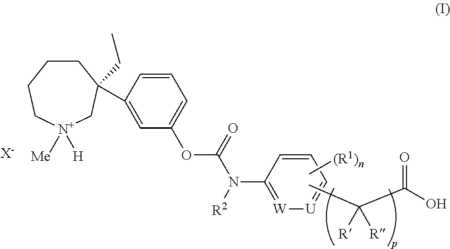

wherein:

R¹ is selected from the group consisting of: hydroxy, carboxy, carboxamido, imino, alkanoyl, cyano, cyanomethyl, nitro, amino, halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy), C₁₋₆ haloalkoxy (e.g. trifluoromethoxy), C₃₋₆ cycloalkyl (e.g. cyclopropyl or cyclohexyl), aryl (e.g. phenyl), aryl-C₁₋₆ alkyl (e.g. benzyl) and C₁₋₆ alkyl aryl;

R² is selected from the group consisting of: H, C₁₋₄ alkyl (e.g. methyl, ethyl or propyl), C₁₋₄ haloalkyl (e.g. trifluoromethyl), C₁₋₄ alkoxy (e.g. methoxy, ethoxy or propoxy), C₁₋₄ haloalkoxy (e.g. trifluoromethoxy);

W and U are each independently selected from the group consisting of: —CH═ and —N═;

R′ and R″ are each independently selected from the group consisting of: H, hydroxy, carboxy, carboxamido, imino, alkanoyl, cyano, cyanomethyl, nitro, amino, halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy), C₁₋₆ haloalkoxy (e.g. trifluoromethoxy), C₃₋₆ cycloalkyl (e.g. cyclopropyl or cyclohexyl), aryl (e.g. phenyl), aryl-C₁₋₆ alkyl (e.g. benzyl) and C₁₋₆ alkyl aryl;

n and p are each independently 0, 1 or 2;

X⁻ is an anion selected from the group comprising: phosphate, maleate, malonate, sulphate, and, methane sulphonate.

According to another aspect, the present invention provides a salt of the present invention for use as a medicament.

According to another aspect, the present invention provides a salt of the present invention for use in the treatment of pain, e.g. neuropathic pain or nociceptive pain.

According to another aspect, the present invention provides a method of treating a disorder in a subject in need thereof with a salt of the present invention. The method comprises orally administering a therapeutically effective amount (e.g., an analgesically effective amount) of the salt of the present invention. The disorder may be one treatable with a meptazinol. For example, the disorder may be pain, e.g. neuropathic pain or nociceptive pain.

Specific types of pain which can be treated with the salts of the present invention include, but are not limited to, acute pain, chronic pain, post-operative pain, pain due to neuralgia (e.g., post herpetic neuralgia or trigeminal neuralgia), pain due to diabetic neuropathy, dental pain, pain associated with arthritis or osteoarthritis, and pain associated with cancer or its treatment. Any of the salts presented herein can be used in a method of treating pain.

The embodiments described herein relate to all aspects of the invention.

In an embodiment, n is 0.

In an alternative embodiment, n is 1.

In an embodiment, n is 1 and R¹ is selected from the group consisting of: hydroxy, nitro, halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy), C₁₋₆ haloalkoxy (e.g. trifluoromethoxy), C₃₋₆ cycloalkyl (e.g. cyclopropyl or cyclohexyl), aryl (e.g. phenyl), aryl-C₁₋₆ alkyl (e.g. benzyl) and C₁₋₆ alkyl aryl. In an embodiment, n is 1 and R¹ is selected from the group consisting of: hydroxy, halogen (e.g. fluoro, chloro or bromo), C₁₋₆ alkyl (e.g. methyl, ethyl or propyl), C₁₋₆ haloalkyl (e.g. trifluoromethyl) and C₁₋₆ alkoxy (e.g. methoxy, ethoxy or propoxy). In an embodiment, n is 1 and R¹ is selected from the group consisting of: hydroxy, C₁₋₆ alkyl (e.g. methyl, ethyl or propyl), C₁₋₆ haloalkyl (e.g. trifluoromethyl) and C₁₋₆ alkoxy (e.g. methoxy, ethoxy or propoxy). In an embodiment, n is 1 and R¹ is yet more selected from the group consisting of: hydroxy, methyl and methoxy.

In an embodiment, W is —CH═. In an alternative embodiment, W is —N═.

In an embodiment, U is —CH═. In an alternative embodiment, U is —N═.

In an embodiment, W is —CH═ and U is —CH═. In an alternative embodiment, W is —CH═ and U is —N═. In an alternative embodiment, W is —N═ and U is —CH═.

In an embodiment, p is 0.

In an alternative embodiment, p is 1 and R′ and R″ are each H.

In an alternative embodiment, R² is selected from the group consisting of: H and C₁₋₄ alkyl (e.g. methyl, ethyl or propyl). Preferably, R² is selected from the group consisting of: H and methyl. In an embodiment, R² is H. In an embodiment, R² is methyl.

The prodrug salt is an acid addition salt of, maleic acid, malonic acid, phosphoric acid, sulphuric acid or methanesulphonic acid.

In an embodiment, the prodrug salt anion X⁻ is a phosphate, maleate, malonate, sulphate or, methane sulphonate salt.

In an embodiment, the prodrug salt is a maleic acid addition salt.

In an embodiment, the prodrug salt is a malonic acid addition salt.

In an embodiment, the prodrug salt is a phosphoric acid addition salt.

In an embodiment, the prodrug salt is a sulphuric acid addition salt.

In an embodiment, the prodrug salt is a methane sulphonic acid addition salt.

In an embodiment, the prodrug salt is of formula (II):

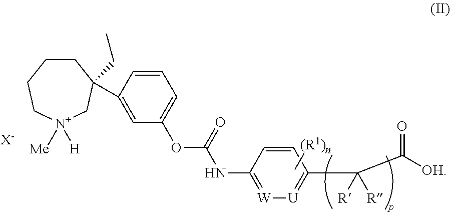

In an embodiment, the prodrug salt is of formula (III):

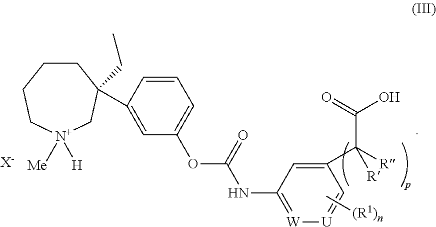

In an embodiment, the prodrug salt is of formula (IV):

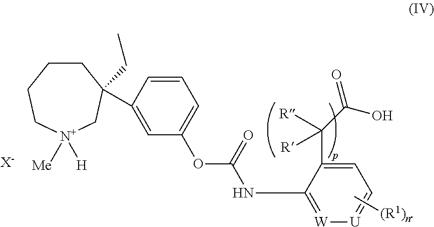

In one embodiment, the prodrug moiety is selected from one of the prodrug moieties provided in Table 1.

TABLE 1
Various prodrugs of the present invention

| | | Structure When Bound to parent drug |
|---|---|---|
| | Prodrug Moiety | (meptazinol) |
| 1 | 2-amino benzoic acid carbamate | 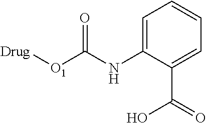 |
| 2 | 3-amino benzoic acid carbamate | 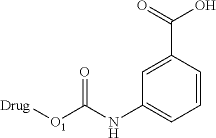 |
| 3 | 4-amino benzoic acid (PABA) carbamate | 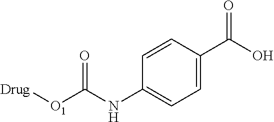 |
| 4 | 4-amino salicylic acid carbamate | 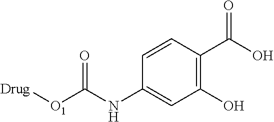 |
| 5 | 4-amino-phenyl acetic acid carbamate | 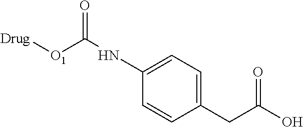 |
| 6 | 4-Amino- 2-Chlorobenzoic Acid carbamate | 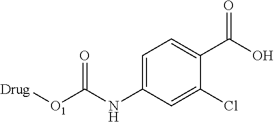 |
| 7 | 6-Aminonicotinic Acid carbamate | 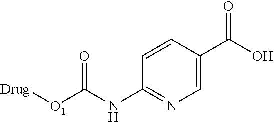 |
| 8 | 2-(4-aminophenyl) propanoic acid carbamate | 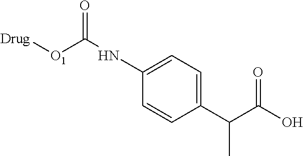 |
| 9 | 4-amino 2-fluorobenzoic acid carbamate | 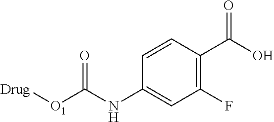 |
| 10 | 4-amino N-methyl benzoic acid carbamate | 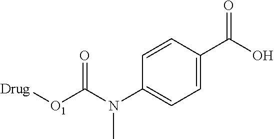 |
| 11 | 4-amino 2-methylbenzoic acid carbamate | 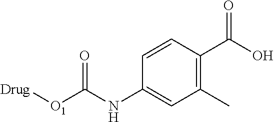 |

In an embodiment, the prodrug salt is of the formula:

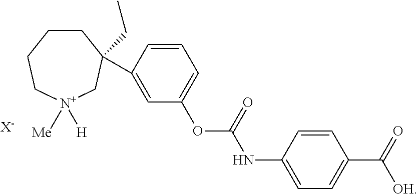

In an embodiment, the prodrug salt is of the formula:

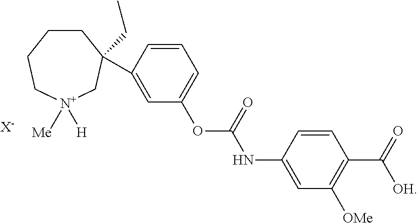

In an embodiment, the prodrug salt is of the formula:

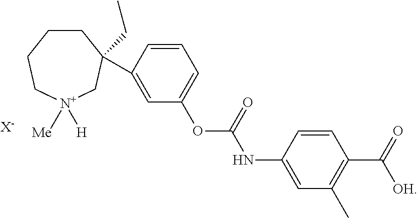

In an embodiment, the prodrug salt is of the formula:

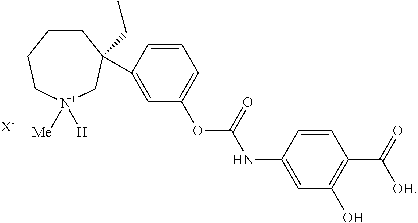

In an embodiment, the prodrug salt is of the formula:

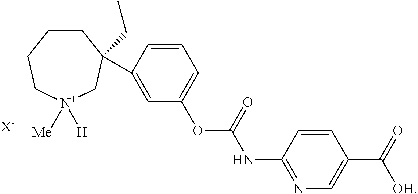

In an embodiment, the prodrug salt is of the formula:

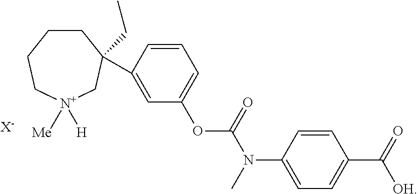

### DETAILED DESCRIPTION OF THE INVENTION

### Definitions

As used herein:

The term “amino” refers to a

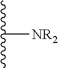

group, wherein each R is independently selected from the group consisting of: H and C₁-C₁₀ alkyl. For example, the term “amino” may refer to a

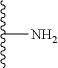

group.

The term “alkyl,” as a group, refers to a straight or branched hydrocarbon chain containing the specified number of carbon atoms. When the term “alkyl” is used without reference to a number of carbon atoms, it is to be understood to refer to a C₁-C₁₀ alkyl, e.g. a C₁, C₂, C₃, C₄, C₅, C₆, C₇, C₈, C₉ or C₁₀ alkyl. For example, C₁₋₁₀ alkyl means a straight or branched saturated hydrocarbon chain containing, for example, at least 1, and at most 10, carbon atoms. Examples of “alkyl” groups, as used herein include, but are not limited to, methyl, ethyl, n-propyl, n-butyl, n-pentyl, isobutyl, isopropyl, t-butyl, hexyl, heptyl, octyl, nonyl and decyl.

The term “alkyl ester,” includes, for example, groups of the formulae

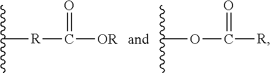

wherein each occurrence of R is independently a straight or branched C₁-C₁₀ alkyl group as defined immediately above.

The term “substituted alkyl” as used herein denotes alkyl radicals wherein at least one hydrogen is replaced by one more substituents such as, but not limited to, hydroxy, alkoxy (for example, C₁-C₁₀ alkoxy, e.g. methoxy or ethoxy), aryl (for example, phenyl), heterocycle, halogen (for example, F, Cl or Br), haloalkyl (for example, C₁-C₁₀ fluoroalkyl, e.g. trifluoromethyl or pentafluoroethyl), cyano, cyanomethyl, nitro, amino (e.g. a

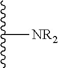

group, wherein each R is independently selected from the group consisting of: H and C₁-C₁₀ alkyl, or a

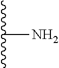

group), amide (e.g., —C(O)NH—R where R is a C₁-C₁₀ alkyl such as methyl), amidine (e.g., —C(═NR)NR₂, wherein each R is independently selected from the group consisting of: H and C₁-C₁₀ alkyl), amido (e.g., —NHC(O)—R where R is a C₁-C₁₀ alkyl such as methyl), carboxamide, carbamate (e.g. —NRC(O)OR, wherein each R is an independently selected C₁-C₁₀ alkyl, e.g. methyl), carbonate (e.g. —C(OR)₃ wherein each R is an independently selected C₁-C₁₀ alkyl, e.g. methyl), ester, alkoxyester (e.g., —C(O)O—R where R is a C₁-C₁₀ alkyl such as methyl) and acyloxyester (e.g., —OC(O)—R where R is a C₁-C₁₀ alkyl such as methyl). The definition pertains whether the term is applied to a substituent itself or to a substituent of a substituent.

The term “cycloalkyl” group as used herein refers to a non-aromatic monocyclic hydrocarbon ring of from 3 to 8 carbon atoms. Exemplary are saturated monocyclic hydrocarbon rings having 1, 2, 3, 4, 5, 6, 7 or 8, carbon atoms such as, for example, cyclopropyl, cyclobutyl, cyclopentyl, cyclohexyl or cycloheptyl.

The term “substituted cycloalkyl” as used herein denotes a cycloalkyl group further bearing one or more substituents as set forth herein, such as those recited in the paragraph defining the substitutents of a “substituted alkyl”. The definition pertains whether the term is applied to a substituent itself or to a substituent of a substituent.

The term “heterocycle” refers to a stable 3- to 15-membered ring radical which consists of carbon atoms and from one to five heteroatoms selected from nitrogen, phosphorus, oxygen and sulphur. For example, a heterocyclic group may be:

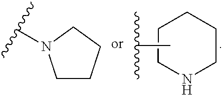

The term “substituted heterocycle” as used herein denotes a heterocycle group further bearing one or more substituents as set forth herein, such as those recited in the paragraph defining the substitutents of a “substituted alkyl”. The definition pertains whether the term is applied to a substituent itself or to a substituent of a substituent. For example, a substituted heterocyclic group may be:

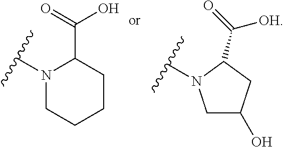

The term “aryl,” as used herein, refers to cyclic, aromatic hydrocarbon groups which have 1 to 3 aromatic rings, for example phenyl or naphthyl. The aryl group may have fused thereto a second or third ring which is a heterocyclo, cycloalkyl, or heteroaryl ring, provided in that case the point of attachment will be to the aryl portion of the ring system. Thus, exemplary aryl groups include

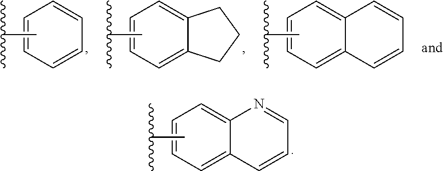

In embodiments, “aryl” refers to a ring structure consisting exclusively of hydrocarbyl groups.

The term “heteroaryl,” as used herein, refers to an aryl group in which at least one of the carbon atoms in the aromatic ring has been replaced by a heteroatom selected from oxygen, nitrogen and sulphur. The nitrogen and/or sulfur heteroatoms may optionally be oxidized and the nitrogen heteroatoms may optionally be quaternized. The heteroaryl group may be a 5 to 6 membered monocyclic, 7 to 11 membered bicyclic, or 10 to 16 membered tricyclic ring system. Thus, exemplary heteroaryl groups include

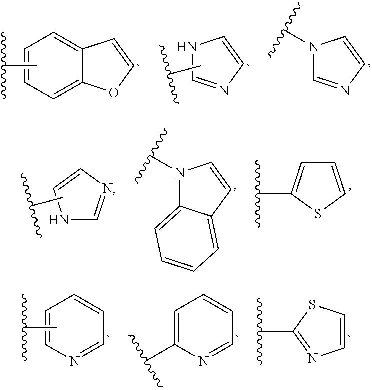

“Substituted aryl” and “substituted heteroaryl” groups refer to either an aryl or heteroaryl group, respectively, substituted by one or more substitutuents at any point of attachment to the aryl or heteroaryl ring (and/or any further ring fused thereto). Exemplary substituents include hydroxy, carboxyl, alkoxy (for example, C₁-C₁₀ alkoxy, e.g. methoxy, ethoxy), aryl, phenyl, heterocycle, halogen (for example F, Cl, Br), haloalkyl (for example, C₁-C₁₀ haloalkyl, e.g. trifluoromethyl or pentafluoroethyl), cyano, cyanomethyl, nitro, amino (e.g. a

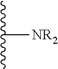

group, wherein each R is independently selected from the group consisting of: H and C₁-C₁₀ alkyl, or a

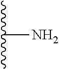

group), amide (e.g., —C(O)NH—R where R is a C₁-C₁₀ alkyl such as methyl), amidine (e.g., —C(═NR)NR₂, wherein each R is independently selected from the group consisting of: H and C₁-C₁₀ alkyl), amido (e.g., —NHC(O)—R where R is a C₁-C₁₀ alkyl such as methyl), carboxamide, carboxylic acid (e.g.,

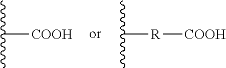

where R is a C₁-C₁₀ alkylene group such as —CH₂—), carbamate (e.g. —NRC(O)OR, wherein each R is an independently selected C₁-C₁₀ alkyl, e.g. methyl), carbonate (e.g. —C(OR)₃ wherein each R is an independently selected C₁-C₁₀ alkyl, e.g. methyl), ester, alkoxyester (e.g., —C(O)O—R where R is a C₁-C₁₀ alkyl such as methyl) and acyloxyester (e.g., —OC(O)—R where R is a C₁-C₁₀ alkyl such as methyl). For example, substituted aryl” and “substituted heteroaryl” groups include:

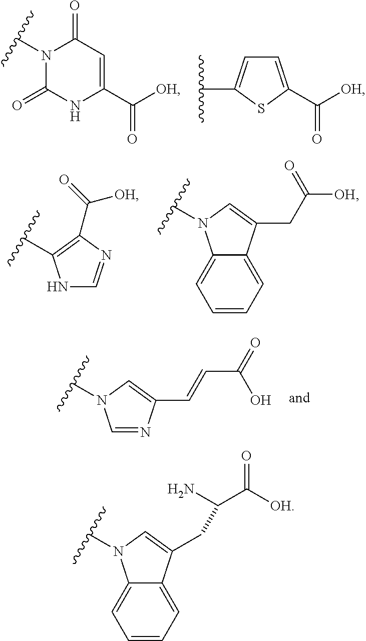

The terms “keto” and “oxo” are synonymous, and refer to the group ═O.

The terms “carbamate group,” “carbamate” and “carbamate linkage” are synonymous, and refer to the group

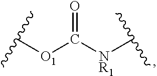

wherein the —O₁— is present in the unbound form of meptazinol.

The term “carrier” refers to a diluent, excipient, and/or vehicle with which an active compound is administered. The pharmaceutical compositions of the invention may contain combinations of more than one carrier. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18^{th} Edition.

The phrase “pharmaceutically acceptable” refers to molecular entities and compositions that are generally regarded as safe. In particular, pharmaceutically acceptable carriers used in the practice of this invention are physiologically tolerable and do not typically produce an allergic or similar untoward reaction (for example, gastric upset, dizziness and the like) when administered to a patient. Preferably, as used herein, the term “pharmaceutically acceptable” means approved by a regulatory agency of the appropriate governmental agency or listed in the U.S. Pharmacopoeia or other generally recognized pharmacopoeia for use in animals, and more particularly in humans.

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the present application includes both one and more than one such excipient.

The term “treating” includes: (1) preventing or delaying the appearance of clinical symptoms of the state, disorder or condition developing in an animal that may be afflicted with or predisposed to the state, disorder or condition but does not yet experience or display clinical or subclinical symptoms of the state, disorder or condition; (2) inhibiting the state, disorder or condition (e.g., arresting, reducing or delaying the development of the disease, or a relapse thereof in case of maintenance treatment, of at least one clinical or subclinical symptom thereof); and/or (3) relieving the condition (i.e., causing regression of the state, disorder or condition or at least one of its clinical or subclinical symptoms). The benefit to a patient to be treated is either statistically significant or at least perceptible to the patient or to the physician.

The term “subject” includes humans and other mammals, such as domestic animals (e.g., dogs and cats).

“Effective amount” means an amount of a prodrug or composition of the present invention sufficient to result in the desired therapeutic response. The therapeutic response can be any response that a user (e.g., a clinician) will recognize as an effective response to the therapy. It is further within the skill of one of ordinary skill in the art to determine appropriate treatment duration, appropriate doses, and any potential combination treatments, based upon an evaluation of therapeutic response.

The term “active ingredient,” unless specifically indicated, is to be understood as referring to the drug portion of a prodrug of the present invention, as described herein.

The term “bioavailability,” as used herein, generally means the rate and extent to which the active ingredient is absorbed from a drug product and becomes systemically available, and hence available at the site of action. See Code of Federal Regulations, Title 21, Part 320.1 (2003 ed.). For oral dosage forms, bioavailability relates to the processes by which the active ingredient is released from the oral dosage form and moves to the site of action. Bioavailability data for a particular formulation provides an estimate of the fraction of the administered dose that is absorbed into the systemic circulation. Thus, the term “oral bioavailability” refers to the fraction of a dose of a respective drug given orally that is absorbed into the systemic circulation after a single administration to a subject. A preferred method for determining the oral bioavailability is by dividing the AUC of the drug given orally by the AUC of the same drug dose given intravenously to the same subject, and expressing the ratio as a percent. Other methods for calculating oral bioavailability will be familiar to those skilled in the art, and are described in greater detail in Shargel and Yu, Applied Biopharmaceutics and Pharmacokinetics, 4th Edition, 1999, Appleton & Lange, Stamford, Conn., incorporated herein by reference in its entirety.

The term “increase in oral bioavailability” refers to the increase in the bioavailability of a respective drug when orally administered as a prodrug of the present invention (either a prodrug compound or composition), as compared to the bioavailability when the drug is orally administered alone. The increase in oral bioavailability can be from 50% to 10,000%, 100% to 10,000%, preferably from 200% to 10,000%, more preferably from 500% to 10,000%, and most preferably from 1000% to 10,000%.

The term “low oral bioavailability,” refers to an oral bioavailability wherein the fraction of a dose of the parent drug given orally that is absorbed into the plasma unchanged after a single administration to a subject is 25% or less, preferably 15% or less, and most preferably 10% or less. Without wishing to be bound by any particular theory, it is believed that the low oral bioavailability of the drugs described herein is the result of the conjugation of a phenolic oxygen to glucuronic acid during first pass metabolism. However, other mechanisms may be responsible for the decrease in oral bioavailability and are contemplated by the present invention.

### Pharmaceutical Compositions of the Invention

While it is possible that, for use in the methods of the invention, the prodrug salt of the present invention may be administered as the isolated substance, the active ingredient may be presented in a pharmaceutical composition, e.g., wherein the agent is in admixture with a pharmaceutically acceptable carrier selected with regard to the intended route of administration and standard pharmaceutical practice. In one embodiment of the present invention, a composition comprising a prodrug of the present invention. The composition comprises at least one prodrug selected from the Formula provided, and at least one pharmaceutically acceptable excipient or carrier.

The formulations of the invention may be immediate-release dosage forms, i.e., dosage forms that release the prodrug at the site of absorption immediately, or controlled-release dosage forms, i.e., dosage forms that release the prodrug over a predetermined period of time. Controlled release dosage forms may be of any conventional type, e.g., in the form of reservoir or matrix-type diffusion-controlled dosage forms; matrix, encapsulated or enteric-coated dissolution-controlled dosage forms; or osmotic dosage forms. Dosage forms of such types are disclosed, e.g., in Remington, The Science and Practice of Pharmacy, 20^{th} Edition, 2000, pp. 858-914.

However, since absorption of prodrugs may proceed via active transporters located in specific regions of GI tract, unconventional controlled dosage forms may be desirable. For example, the Pept1 transporter is believed to be largely confined to the upper GI tract, and should it be a contributor to prodrug absorption, may limit the effectiveness for continued absorption along the whole length of the GI tract.

For those prodrugs which do not result in sustained plasma drugs levels due to continuous generation of active agent from a plasma reservoir of prodrug—but which may offer other advantages—gastroretentive or mucoretentive formulations analogous to those used in metformin products such as Glumetz® or Gluphage XR® may be useful. The former exploits a drug delivery system known as Gelshield Diffusion™ Technology while the latter uses a so-called Acuform™ delivery system. In both cases the concept is to retain drug in the stomach, slowing drug passage into the ileum maximizing the period over which absorption take place and effectively prolonging plasma drug levels. Other drug delivery systems affording delayed progression along the GI tract, such as mucoadhesive formulations, may also be of value.

The formulations of the present invention can be administered, for example, from one to six times daily, depending on the dosage form and dosage.

The prodrug employed in the present invention may be used in combination with other therapies and/or active agents.

In the methods of treating pain, the prodrugs may be administered in conjunction with other therapies and/or in combination with other active agents (e.g., other analgesics). For example, the salts may be administered to a subject in combination with other active agents used in the management of pain. An active agent to be administered in combination with the salts encompassed by the present invention may include, for example, a drug selected from the group consisting of non-steroidal anti-inflammatory drugs (e.g., ibuprofen), anti-emetic agents (e.g., ondansetron, domerperidone, hyoscine and metoclopramide), or unabsorbed or poorly bioavailable opioid antagonists (e.g., naloxone) to reduce the risk of drug abuse. In such combination therapies, the salts encompassed by the present invention may be administered prior to, concurrent with, or subsequent to the other therapy and/or active agent. The salt and other active agent(s) may also be incorporated into a single dosage form.

When combined in the same formulation, it will be appreciated that the two compounds must be stable and compatible with each other and the other components of the formulation. When formulated separately, they may be provided in any convenient formulation, conveniently in such manner as are known for such compounds in the art.

The prodrugs presented herein may be formulated for administration in any convenient way for use in human or veterinary medicine. The invention therefore includes pharmaceutical compositions comprising a compound of the invention adapted for use in human or veterinary medicine. Such compositions may be presented for use in a conventional manner with the aid of one or more suitable carriers. Acceptable carriers for therapeutic use are well-known in the pharmaceutical art, and are described, for example, in Remington's Pharmaceutical Sciences, Mack Publishing Co. (A. R. Gennaro edit. 1985). The choice of pharmaceutical carrier can be selected with regard to the intended route of administration and standard pharmaceutical practice. The pharmaceutical compositions may comprise as, in addition to, the carrier any suitable binder(s), lubricant(s), suspending agent(s), coating agent(s), and/or solubilizing agent(s).

Preservatives, stabilizers, dyes and even flavoring agents may be provided in the pharmaceutical composition. Examples of preservatives include sodium benzoate, ascorbic acid and esters of p-hydroxybenzoic acid. Antioxidants and suspending agents may also be used.

The compounds used in the invention may be milled using known milling procedures such as wet milling to obtain a particle size appropriate for tablet formation and for other formulation types. Finely divided (nanoparticulate) preparations of the compounds may be prepared by processes known in the art, see, e.g., International Patent Application No. WO 02/00196 (SmithKline Beecham).

The compounds and pharmaceutical compositions of the present invention are intended to be administered orally (e.g., as a tablet, sachet, capsule, pastille, pill, bolus, powder, paste, granules, bullets or premix preparation, ovule, elixir, solution, suspension, dispersion, gel, syrup or as an ingestible solution). In addition, compounds may be present as a dry powder for constitution with water or other suitable vehicle before use, optionally with flavoring and coloring agents. Solid and liquid compositions may be prepared according to methods well-known in the art. Such compositions may also contain one or more pharmaceutically acceptable carriers and excipients which may be in solid or liquid form.

Dispersions can be prepared in a liquid carrier or intermediate, such as glycerin, liquid polyethylene glycols, triacetin oils, and mixtures thereof. The liquid carrier or intermediate can be a solvent or liquid dispersive medium that contains, for example, water, ethanol, a polyol (e.g., glycerol, propylene glycol or the like), vegetable oils, non-toxic glycerine esters and suitable mixtures thereof. Suitable flowability may be maintained, by generation of liposomes, administration of a suitable particle size in the case of dispersions, or by the addition of surfactants.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia.

Additionally, lubricating agents such as magnesium stearate, stearic acid, glyceryl behenate and talc may be included.

Examples of pharmaceutically acceptable disintegrants for oral compositions useful in the present invention include, but are not limited to, starch, pre-gelatinized starch, sodium starch glycolate, sodium carboxymethylcellulose, croscarmellose sodium, microcrystalline cellulose, alginates, resins, surfactants, effervescent compositions, aqueous aluminum silicates and crosslinked polyvinylpyrrolidone.

Examples of pharmaceutically acceptable binders for oral compositions useful herein include, but are not limited to, acacia, cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthane resin, alginates, magnesium-aluminum silicate, polyethylene glycol or bentonite.

Examples of pharmaceutically acceptable fillers for oral compositions useful herein include, but are not limited to, lactose, anhydrolactose, lactose monohydrate, sucrose, dextrose, mannitol, sorbitol, starch, cellulose (particularly microcrystalline cellulose), dihydro- or anhydro-calcium phosphate, calcium carbonate and calcium sulfate.

Examples of pharmaceutically acceptable lubricants useful in the compositions of the invention include, but are not limited to, magnesium stearate, talc, polyethylene glycol, polymers of ethylene oxide, sodium lauryl sulfate, magnesium lauryl sulfate, sodium oleate, sodium stearyl fumarate, and colloidal silicon dioxide.

Examples of suitable pharmaceutically acceptable odorants for the oral compositions include, but are not limited to, synthetic aromas and natural aromatic oils such as extracts of oils, flowers, fruits (e.g., banana, apple, sour cherry, peach) and combinations thereof, and similar aromas. Their use depends on many factors, the most important being the organoleptic acceptability for the population that will be taking the pharmaceutical compositions.

Examples of suitable pharmaceutically acceptable dyes for the oral compositions include, but are not limited to, synthetic and natural dyes such as titanium dioxide, beta-carotene and extracts of grapefruit peel.

Examples of useful pharmaceutically acceptable coatings for the oral compositions, typically used to facilitate swallowing, modify the release properties, improve the appearance, and/or mask the taste of the compositions include, but are not limited to, hydroxypropylmethylcellulose, hydroxypropylcellulose and acrylate-methacrylate copolymers.

Suitable examples of pharmaceutically acceptable sweeteners for the oral compositions include, but are not limited to, aspartame, saccharin, saccharin sodium, sodium cyclamate, xylitol, mannitol, sorbitol, lactose and sucrose.

Suitable examples of pharmaceutically acceptable buffers useful herein include, but are not limited to, citric acid, sodium citrate, sodium bicarbonate, dibasic sodium phosphate, magnesium oxide, calcium carbonate and magnesium hydroxide.

Suitable examples of pharmaceutically acceptable surfactants useful herein include, but are not limited to, sodium lauryl sulfate and polysorbates.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

Suitable examples of pharmaceutically acceptable preservatives include, but are not limited to, various antibacterial and antifungal agents such as solvents, for example ethanol, propylene glycol, benzyl alcohol, chlorobutanol, quaternary ammonium salts, and parabens (such as methyl paraben, ethyl paraben, propyl paraben, etc.).

Suitable examples of pharmaceutically acceptable stabilizers and antioxidants include, but are not limited to, ethylenediaminetetriacetic acid (EDTA), thiourea, tocopherol and butyl hydroxyan

The pharmaceutical compositions of the invention may contain from 0.01 to 99% weight per volume of the prodrugs encompassed by the present invention.

### Dosages

The doses referred to throughout the specification refer to the amount of the free base equivalents in the particular compound, unless otherwise specified.

Appropriate patients to be treated according to the methods of the invention include any human or animal in need of treatment. The patient is preferably a mammal, more preferably a human, but can be any subject or animal, including a laboratory animal in the context of a clinical trial, screening, or activity experiment employing an animal model. Thus, as can be readily appreciated by one of ordinary skill in the art, the methods and compositions of the present invention are particularly suited to administration to any animal or subject, particularly a mammal, and including, but not limited to, domestic animals, such as feline or canine subjects, farm animals, such as but not limited to bovine, equine, caprine, ovine, and porcine subjects, research animals, such as mice, rats, rabbits, goats, sheep, pigs, dogs, cats, etc., avian species, such as chickens, turkeys, songbirds, etc.

Typically, a physician will determine the actual dosage which will be most suitable for an individual subject. The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

Depending on the severity of condition to be treated, a suitable therapeutically effective and safe dosage, as may readily be determined within the skill of the art, can be administered to subjects. For oral administration to humans, the daily dosage level of the prodrug may be in single or divided doses. The duration of treatment may be determined by one of ordinary skill in the art, and should reflect the nature of the condition (e.g., a chronic versus an acute condition) and/or the rate and degree of therapeutic response to the treatment. Typically, a physician will determine the actual dosage which will be most suitable for an individual subject.

The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. For highly potent agents, the daily dose requirement may, for example, range from 0.5 to 50 mg, e.g. from 1 to 25 mg, and optionally from 1 mg to 10 mg (all with reference to the free base content). For less potent agents, the daily dose requirement may, for example, range from 1 mg to 1600 mg, e.g. from 1 mg to 800 mg and optionally from 1 mg to 400 mg.

In the methods of treatment, the prodrugs encompassed by the present invention may be administered in conjunction with other therapies and/or in combination with other active agents. In such combination therapies, the prodrugs encompassed by the present invention may be administered prior to, concurrent with, or subsequent to the other therapy and/or active agent.

Where the prodrugs encompassed by the present invention are administered in conjunction with another active agent, the individual components of such combinations may be administered either sequentially or simultaneously in separate or combined pharmaceutical formulations by any convenient route. When administration is sequential, either the prodrugs encompassed by the present invention or the second active agent may be administered first. For example, in the case of a combination therapy with another active agent, the prodrugs encompassed by the present invention may be administered in a sequential manner in a regimen that will provide beneficial effects of the drug combination. When administration is simultaneous, the combination may be administered either in the same or different pharmaceutical composition. For example, a prodrug encompassed by the present invention and another active agent may be administered in a substantially simultaneous manner, such as in a single capsule or tablet having a fixed ratio of these agents, or in multiple separate dosage forms for each agent.

When the prodrugs of the present invention are used in combination with another agent active in the methods for treatment, the dose of each compound may differ from that when the compound is used alone. Appropriate doses will be readily appreciated by those of ordinary skill in the art.

### EXAMPLES

### Example 1

### Meptazinol PABA Carbamate Malonic Acid Addition Salt

Meptazinol PABA carbamate (15.00 g, 37.8 mmol) and malonic acid (3.94 g, 37.8 mmol) were dissolved in methanol (150 ml) giving a clear solution, to which was added tert-butyl methyl ether (1.5 L). An oil phase and clear supernatant were formed and this mixture was stirred at 15-25° C. for 15 hours. The supernatant was isolated from the mixture and concentrated to dryness. The concentrated residue and the oil phase were added to methanol (150 ml) and to the turbid mixture was added tert-butyl methyl ether (1.5 L) and the mixture stirred at 15-25° C. for 15 hours. The solid was isolated by filtration, under an atmosphere of nitrogen, and dried in vacuo at 40° C. to afford the title compound (13.96 g, 74% th yield).

NMR Spectrum (d₆-DMSO)

10.6 (s, 1H, carbamate NH), 7.9 (d, 2H, ArH), 7.6 (d, 2H, ArH), 7.5 (t, 1H, ArH), 7.3 (b, 2H, ArH), 7.2 (d, 1H, ArH), 3.6-2.8 (b, 4H, CH₂), 2.9 (s, 2H, CH₂ malonic acid), 2.6 (s, 3H, CH₃), 2.3-1.6 (b, 8H, CH₂), 0.5 (t, 3H, CH₃).

### Example 2

### Meptazinol PABA Carbamate Maleic Acid Addition Salt

Meptazinol PABA carbamate (1.02 g, 2.6 mmol) and maleic acid (0.30 g, 2.6 mmol) were dissolved in methanol (10 ml) giving a clear solution, to which was added tert-butyl methyl ether (100 ml). The mixture was stirred at 15-25° C. for 3 days, the solid formed isolated by filtration, under an atmosphere of nitrogen, and dried in vacuo at 40° C. to afford the title compound (1.07 g, 82% th yield).

NMR Spectrum (d₆-DMSO)

10.6 (s, 1H, carbamate NH), 8.5 (b, 1H, COOH), 7.9 (d, 2H, ArH), 7.6 (d, 2H, ArH), 7.5 (t, 1H, ArH), 7.3 (b, 2H, ArH), 7.2 (d, 1H, ArH), 6.1 (s, 2H, CH maleic acid), 4.0-3.1 (b, 4H, CH₂), 2.8 (s, 3H, CH₃), 2.3-1.6 (b, 8H, CH₂), 0.5 (t, 3H, CH₃).

### Example 3

### Meptazinol PABA Carbamate Phosphoric Acid Addition Salt

Meptazinol PABA carbamate (1.01 g, 2.6 mmol) and phosphoric acid (0.25 g, 2.6 mmol) were dissolved in methanol (10 ml) giving a clear solution, to which was added methyl ethyl ketone (100 ml). The mixture was stirred at 15-25° C. for 3 days, the solid formed isolated by filtration, under an atmosphere of nitrogen, and dried in vacuo at 40° C. to afford the title compound (1.01 g, 87% th yield).

NMR Spectrum (d₆-DMSO)

10.6 (s, 1H, carbamate NH), 7.9 (d, 2H, ArH), 7.6 (d, 2H, ArH), 7.5 (t, 1H, ArH), 7.3 (b, 2H, ArH), 7.2 (d, 1H, ArH), 3.3-2.5 (b, 4H, CH₂), 2.4 (s, 3H, CH₃), 2.2-1.6 (b, 8H, CH₂), 0.5 (t, 3H, CH₃).

### Example 4

### Meptazinol PABA Carbamate Sulphuric Acid Addition Salt

Meptazinol PABA carbamate (1.03 g, 2.6 mmol) and sulphuric acid (0.26 g, 2.6 mmol) were dissolved in methanol (10 ml) giving a clear solution, to which was added tert-butyl methyl ether (100 ml). The mixture was stirred at 15-25° C. for 3 days, the solid formed isolated by filtration, under an atmosphere of nitrogen, and dried in vacuo at 40° C. to afford the title compound (1.14 g, 89% th yield).

NMR Spectrum (d₆-DMSO)

10.6 (s, 1H, carbamate NH), 8.5 (b, 1H, COOH), 7.9 (d, 2H, ArH), 7.6 (d, 2H, ArH), 7.5 (t, 1H, ArH), 7.3 (b, 2H, ArH), 7.2 (d, 1H, ArH), 4.0-3.1 (b, 4H, CH₂), 2.8 (s, 3H, CH₃), 2.3-1.4 (b, 8H, CH₂), 0.5 (t, 3H, CH₃).

### Example 5

### Meptazinol PABA Carbamate Methanesulphonic Acid Addition Salt

Meptazinol PABA carbamate (1.01 g, 2.6 mmol) and methanesulphonic acid (0.24 g, 2.6 mmol) were dissolved in methanol (10 ml) giving a clear solution, to which was added toluene (100 ml). The mixture was stirred at 15-25° C. for 3 days, the solid formed isolated by filtration, under an atmosphere of nitrogen, and dried in vacuo at 40° C. to afford the title compound (1.02 g, 81% th yield).

NMR Spectrum (d₆-DMSO)

10.6 (s, 1H, carbamate NH), 8.5 (b, 1H, COON), 7.9 (d, 2H, ArH), 7.6 (d, 2H, ArH), 7.5 (t, 1H, ArH), 7.3 (b, 2H, ArH), 7.2 (d, 1H, ArH), 3.7-3.1 (b, 4H, CH₂), 2.8 (s, 3H, CH₃), 2.4 (s, 3H, CH₃, methanesulphonic acid), 2.5-1.5 (b, 8H, CH₂), 0.5 (t, 3H, CH₃).

### Example 6

### Meptazinol PABA Carbamate Maleic Acid Addition Salt (Large Scale Method)

Meptazinol PABA carbamate (24.60 Kg, 62.1 mol), 2-propanol (78.6 Kg) and water (12.5 L) were charged to an appropriate sized vessel (Vessel A) and the temperature of the agitated mixture adjusted to 18 to 23° C. Maleic acid (7.1 Kg, 61.2 mol), 2-propanol (77.1 Kg) and water (12.5 L) were charged to an appropriate sized vessel (Vessel B) and the temperature of the agitated mixture adjusted to 18 to 23° C. The contents of Vessel B were charged to Vessel A over a period of 84 minutes maintaining the temperature at 18 to 23° C., followed by a pre-mixed solution of 2-propanol (17.2 Kg) and water (2.5 L).

The contents of Vessel A were stirred at 18 to 23° C. for 14 hours, seed crystals of SSP-1302 maleate salt were added and the mixture stirred for a further 3 hours. The mixture was cooled to 0 to 5° C. over a period of 27 minutes and then stirred at this temperature for 17 hours. The mixture was then warmed to 18 to 23° C. over a period of 30 minutes followed by the addition of tert-butyl methyl ether (126 L) over a period of 60 minutes and the resulting slurry stirred at 18 to 23° C. for 6 hours.

The mixture was filtered, the filter cake washed with tert-butyl methyl ether (91.3 L) and the solid dried in vacuo ≦40° C. to afford the title compound (23.04 Kg, 72% th yield)

NMR Spectrum (d₆-DMSO)

10.6 (s, 1H, carbamate NH), 8.5 (b, 1H, COON), 7.9 (d, 2H, ArH), 7.6 (d, 2H, ArH), 7.5 (t, 1H, ArH), 7.3 (b, 2H, ArH), 7.2 (d, 1H, ArH), 6.1 (s, 2H, CH maleic acid), 4.0-3.1 (b, 4H, CH₂), 2.8 (s, 3H, CH₃), 2.3-1.6 (b, 8H, CH₂), 0.5 (t, 3H, CH₃).

### Example 7

### Physicochemical Properties of the Salts According to the Invention

The advantageous properties of salts according to the invention can be seen from the table below:

| | Easy | Improvement | | Good |
|---|---|---|---|---|
| | to | in chemical | | thermal |
| | make/ | purity after | | profile |
| | high | preparation | Hygro- | with clean |
| Compound | yield | vs. input | scopic | melt event |
| meptazinol | — | — | yes | no |
| p-amino-benzoylcarbamate | | | | |
| meptazinol p-amino- | yes | yes | no | yes |
| benzoylcarbamate•Malonic | | | | |
| acid addition salt | | | | |
| meptazinol p-amino- | yes | yes | no | no |
| benzoylcarbamate•Phos- | | | | |
| phoric acid addition salt | | | | |
| meptazinol p-amino- | yes | yes | no | yes |
| benzoylcarbamate•Maleic | | | | |
| acid addition salt | | | | |
| meptazinol p-amino- | yes | yes | no | yes |
| benzoylcarbamate•Sul- | | | | |
| phuric acid addition salt | | | | |
| meptazinol p-amino- | yes | yes | no | yes |
| benzoylcarbamate•Meth- | | | | |
| anesulphonic acid | | | | |
| addition salt | | | | |
| meptazinol p-amino- | yes | yes | yes | no |
| benzoylcarbamate•HCl | | | | |

As can be seen from the above table, the isolated salts were all obtained in chemical purities which were higher than the free base starting material. The salts were all obtained in good yields. Additionally, the maleic acid, malonic acid, phosphoric acid, sulphuric acid and methanesuiphonic acid addition salts were found to not be hygroscopic. The maleic acid, malonic acid, sulphuric acid and methanesulphonic acid addition salts all exhibited good thermal profiles with clean melt events.

Patents, patent applications, and non-patent literature cited in herein are hereby incorporated by reference in their entirety.

1. A salt of a meptazinol prodrug of formula (I): 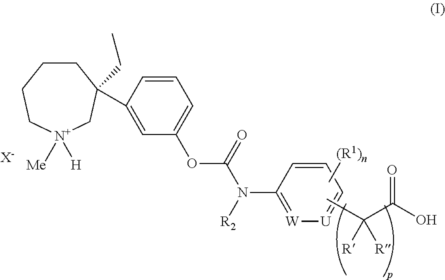
wherein:
R¹ is selected from the group consisting of hydroxy, carboxy, carboxamido, imino, alkanoyl, cyano, cyanomethyl, nitro, amino, halogen, C₁₋₆ alkyl, C₁₋₆ haloalkyl, C₁₋₆ alkoxy, C₁₋₆ haloalkoxy, C₃₋₆ cycloalkyl, aryl, aryl-C₁₋₆ alkyl and C₁₋₆ alkyl aryl;
R² is selected from the group consisting of: H, C₁₋₄ alkyl, C₁₋₄ haloalkyl, C₁₋₄ alkoxy, C₁₋₄ haloalkoxy;
W and U are each independently selected from the group consisting of: —CH═ and —N═;
R′ and R″ are each independently selected from the group consisting of: H, hydroxy, carboxy, carboxamido, imino, alkanoyl, cyano, cyanomethyl, nitro, amino, halogen, C₁₋₆ alkyl, C₁₋₆ haloalkyl, C₁₋₆ alkoxy, C₁₋₆ haloalkoxy, C₃₋₆ cycloalkyl, aryl, aryl-C₁₋₆ alkyl and C₁₋₆ alkyl aryl;
n and p are each independently 0, 1 or 2;
X⁻ is an anion selected from the group comprising: phosphate, maleate, malonate, sulphate, and methane sulphonate.

2. The salt according to claim 1 wherein R² is selected from the group consisting of: H and C₁₋₄ alkyl.

3. The salt according to claim 1 of formula (II):

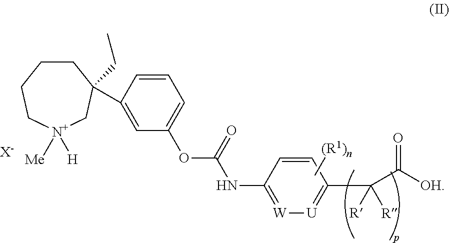

4. The salt according to claim 1 wherein n is 0.

5. The salt according to claim 1 wherein n is 1.

6. The salt according to claim 5 wherein R¹ is selected from the group consisting of: hydroxy, halogen, C₁₋₆ alkyl, C₁₋₆ haloalkyl and C₁₋₆ alkoxy.

7. The salt according to claim 6 wherein R¹ is selected from the group consisting of hydroxy, methyl and methoxy.

8. The salt according to claim 1 wherein W is —CH═.

9. The salt according to claim 1 wherein U is —CH═.

10. The salt according to claim 1 wherein p is 0.

11. The salt according to claim 1 wherein p is 1 and R′ and R″ are each H.

12. The salt according to claim 1 wherein the meptazinol prodrug has the formula:

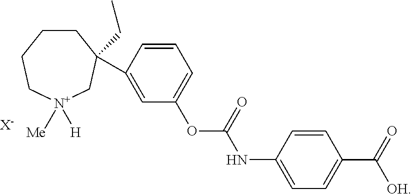

13. Use of a salt according to claim 1 for the manufacture of a medicament.

14. Use of claim 13, wherein the medicament is for the treatment of pain.

15. A pharmaceutical composition comprising a salt according to claim 1 and at least one pharmaceutically acceptable carrier.

16. A method for of treating a patient having a pain disorder, comprising orally administering to the patient in need thereof a therapeutically effective amount of the salt according to claim 1.

17. (canceled)

18. The method according to claim 16, wherein the pain disorder is neuropathic or nociceptive pain.

19. Use of claim 14, wherein the pain is neuropathic or nociceptive pain.
